(19) United States
(12) Patent Application Publication
(10) Pub. No.: US 2009/0043369 A1
(43) Pub. Date: Feb. 12, 2009

(54) Flexible Biodegradable Coating For Implantable Medical Devices

(75) Inventor: Andreas Radeloff, Frankfurt (DE)

Correspondence Address: BROMBERG & SUNSTEIN LLP, 125 SUMMER STREET, BOSTON, MA, 02110-1618, US

(73) Assignee: MED-EL Elektromedizinische Geraete GmbH, Innsbruck (AT)

(21) Appl. No.: 12/129,841

(22) Filed: May 30, 2008

Related U.S. Application Data
(60) Provisional application No. 60/941,320, filed on Jun. 1, 2007.

(51) Int. Cl. A61N 1/00; A61F 13/00

(52) U.S. Cl. 607/137; 424/422

(57) ABSTRACT

An implantable device includes an implantable cochlear electrode carrier for electrically stimulating cochlear tissue. A flexible lubricant coating covers the outer surface of at least a portion of the electrode carrier and may include a therapeutically effective amount of a pharmaceutical agent for surrounding tissue of the inner ear.

This application claims priority from U.S. Provisional Patent Application 60/941,320, filed Jun. 1, 2007, which is incorporated herein by reference.

### FIELD OF THE INVENTION

The present invention relates to implantable medical devices, and more specifically, to a therapeutic coating for such devices.

### BACKGROUND ART

Implantable medical devices include cochlear implants (CI) in which electrode carriers are inserted into the inner ear of deaf and profoundly hearing impaired patients to electrically stimulate nerve fibers. Preferably, friction between the electrode carrier and the inner ear should be minimized in order to avoid trauma to the inner ear. This may preserve some residual hearing in the patient. But electrode carriers are formed of elastomers such as silicone which is water-repellent and creates significant friction on epithelium of the inner ear.

Hyaluronic acid is commonly used as a lubricant that is applied to the electrode carrier immediately prior to insertion. It is not an ideal lubricant because it is similar to water and therefore repelled by the silicone of the electrode carrier. A good lubricant, by contrast, should build a film on both the silicone of the electrode carrier and the epithelium of the inner ear. Other commonly used lubrication coatings (for example, based on polyethylene glycol (PEG) or cellulose derivates) are inflexible and build a rigid film on the silicone. Therefore, they are not suitable for CI electrode carriers.

Sterile inflammatory reaction and soft tissue formation are commonly seen following implantation of an electrode carrier in the inner ear. This seems to account for a need for higher stimulation currents, and may also play an important role in hearing loss since the hair cells are postmitotic so that inflammation causes dysfunctional scar tissue. Inhibitors of apoptosis pathways have been suggested for use in acute hearing disorders. Steroids are commonly used in ear surgery and cochlear implantation since they are thought to have a protective effect on hair cells and hearing function.

Local application of therapeutic substances to the inner ear is discussed for a variety of issues related to cochlear implantation, e.g., apoptosis inhibitors to preserve residual hearing and neurotrophins to attract neurons to the electrodes. Pharmaceutical agents are applied in various different ways such as systemically, to the round window, and/or through the round window. Among the disadvantages are systemic side effects and low concentration in apical parts of the cochlea.

Drug action may be needed in apical parts of the cochlea where the residual hair cells are located. It is known that in systemic application and application to the round window or the basal part of the cochlea, drug concentration is very low in the apical parts. This is described for example in Mynatt R. et al., Demonstration Of A Longitudinal Concentration Gradient Along Scala Tympani By Sequential Sampling Of Perilymph From The Cochlear Apex, J Assoc Res Otolaryngol. 2006 Jun; 7(2):182-93, which is incorporated herein by reference.

### SUMMARY OF THE INVENTION

Embodiments of the present invention include an implantable device such as an implantable cochlear electrode carrier for electrically stimulating cochlear tissue. A flexible lubricant coats the outer surface of at least a portion of the electrode carrier for reducing friction between the electrode carrier and epithelium.

In some specific embodiments, the coating may be based on a polymer containing a wetting agent for maintaining flexibility of the electrode carrier. For example, the polymer may include hyaluronic acid, hydroxyethyl cellulose (HEC), carboxymethyl cellulose (CMC), hydroxyethyl starch, polyethylene glycol (PEG) or carbomer. The wetting agent may include glycerol or propylene glycol. The coating also may be biodegradable.

In an embodiments, the portion of the electrode carrier may specifically be the apical end, or it may includes the entire insertable portion of the electrode carrier. In addition or alternatively, the thickness of the coating may vary along the length of the electrode carrier.

In some embodiments, the coating includes a therapeutically effective amount of a pharmaceutical agent for surrounding tissue of the inner ear. The concentration of the pharmaceutical agent may vary along the length of the electrode carrier. The pharmaceutical agent may include a steroid, a neurotrophin, or an apoptosis inhibitor. In addition or alternatively, the pharmaceutical agent may include an immunosuppressive component or an anti-proliferative component. The coating may include a first region having a first pharmaceutical agent and a second region having a second pharmaceutical agent.

Embodiments of the present invention also include a lubricant coating for an implantable medical device. The coating includes a flexible lubricant component adapted for coating the outer surface of at least a portion of a cochlear implant electrode carrier for reducing friction between the electrode carrier and epithelium.

In specific such embodiments, the flexible lubricant component may be based on a polymer containing a wetting agent for maintaining flexibility of the electrode carrier. For example, the polymer may include hyaluronic acid, hydroxyethyl cellulose (HEC), carboxymethyl cellulose (CMC), hydroxyethyl starch, or carbomer. The wetting agent may include glycerol or propylene glycol. The coating also may be biodegradable.

In some embodiments, the coating may include a therapeutic component including therapeutically effective amount of a pharmaceutical agent for surrounding tissue of the electrode carrier when implanted. The pharmaceutical agent may include an immunosuppressive component or an anti-proliferative component. The coating may include a first therapeutic component having a first pharmaceutical agent and a second therapeutic component having a second pharmaceutical agent.

### BRIEF DESCRIPTION OF THE DRAWINGS

FIG. 1 shows an example of an implantable medical device according to one specific embodiment of the present invention

### DETAILED DESCRIPTION OF SPECIFIC EMBODIMENTS

Embodiments of the present invention are directed to a coating for the outer surface of implantable medical devices such as a cochlear implant (CI) electrode carrier. The coating is (i) lubricating, (ii) flexible, and (iii) therapeutic. The lubricant quality of the coating reduces friction as the device is inserted into tissue. Reducing friction in turn reduces trauma to the tissue and improves the ability to insert the device. The flexible quality of the coating works together with the lubricating action to be especially useful for a flexible device such as a CI electrode carrier. The therapeutic quality of the coating derives from incorporation of one or more pharmaceutical agents (i.e. drugs) into the substance of the coating for post-insertion release into the surrounding tissue such as the perilymph.

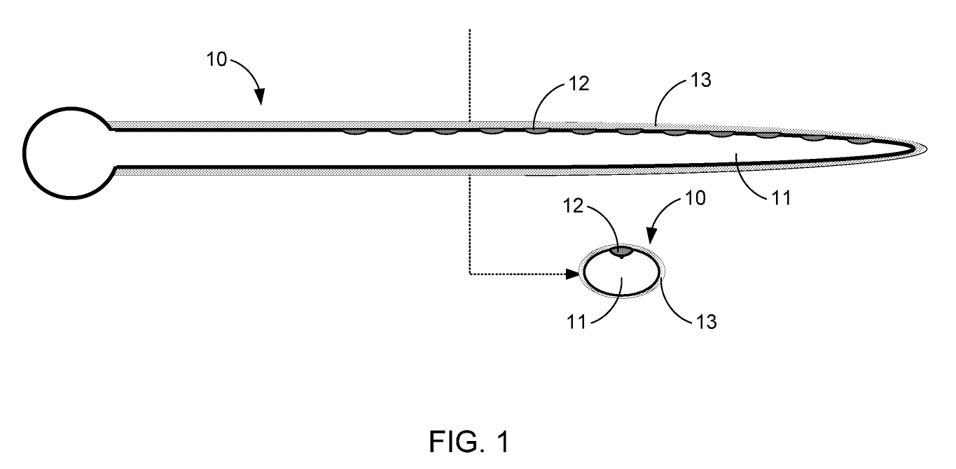

FIG. 1 shows an example of an implantable cochlear prosthesis 10 according to one specific embodiment of the present invention. An elastomeric electrode carrier 11 (e.g. of molded silicone) includes multiple stimulator electrodes 12 for electrically stimulating cochlear tissue. A flexible lubricant coating 13 coats the outer surface of at least a portion of the electrode carrier 11 and includes a therapeutically effective amount of a pharmaceutical agent for surrounding tissue of the inner ear. In addition, the coating 13 may be biodegradable so that after insertion and release of its pharmaceutical agent into surrounding tissue, the coating decomposes and is removed by natural biological processes.

The coating 13 may be based, for example, on a polymer containing a wetting agent for maintaining flexibility of the electrode carrier. For example, the polymer may include hyaluronic acid, hydroxyethyl cellulose (HEC), carboxymethyl cellulose (CMC), hydroxyethyl starch, polyethylene glycol (PEG) or carbomer. The wetting agent may include glycerol or propylene glycol.

The concentration of the pharmaceutical agent in the coating 13 may need to be relatively high to be effective in the fluids of the inner ear, especially in the more apical parts of the cochlea where the still functioning hair cells reside. It may be difficult to release effective levels of the pharmaceutical agent systemically or locally to the basal parts of the cochlea without some sort of significant gradient in concentration from basal to apical parts of the cochlea. Overall, a low systemic release of the pharmaceutical agent is expected, and therefore, low side effects.

In clinical practice and animal models, inflammatory reaction and tissue proliferation has been observed. In such circumstances examples of pharmaceutical agents which may be usefully incorporated into the coating 13 may include immunosuppressive and antiproliferative agents. These drugs may have significant side effects when used systemically, but are virtually free of side effects when used locally. Therefore, antiproliferatives, such as Sirolimus and Methotrexate, and of immunosuppressives, such as Cyclosporin, Tacrolimus and Etanercept, may be useful in specific embodiments.

In some specific embodiments, the coating 13 may be confined mainly to the apical end of the electrode carrier 11 most of the residual hair cells reside. In other embodiments, the coating 13 may cover most or all of the insertable portion of the electrode carrier 11. For example, the coating 13 may extend from the apical end of the electrode carrier 11 to a point 180° after the basal end following insertion into the cochlea-in this range a high friction during insertion is expected. On the other hand, the uncoated non-lubricated portion of the electrode carrier 11 (especially towards the basal end) is likely to be easier for a surgeon to handle.

Besides varying in the specific portion of the electrode carrier 11 which is covered, the concentration of the pharmaceutical agent in the coating 13 may vary; for example, along the length of the electrode carrier. In addition or alternatively, the thickness of the coating 13 may vary along the length of the electrode carrier 11. In some embodiments, the coating 13 may include a first region having a first pharmaceutical agent and a second region having a second pharmaceutical agent.

The coating 13 may be applied to the electrode carrier 11 in various ways. for example, the electrode carrier 11 may be immersed into a container of the coating material. Or the coating material may be sprayed onto the surface of the electrode carrier 11. Masking may be used to confine the coating 13 to the desired portion of the surface of the electrode carrier 11.

For example, in one embodiment the coating 13 is based on a solution of 0.5 to 1.0% of hydroxyethyl cellulose (HEC) in a mixture of aqua ad injectabilia and ethanol in a ratio of 1:2 to which 5-10% glycerol is added. A suitable quantity of a pharmaceutical agent is added to this mixture and the electrode carrier 11 can be dipped into it and air dried at a temperature of approx. 60-80° C. to form the coating 13. The procedure can be repeated as necessary to cover different portions of the electrode carrier 11 or apply different pharmaceutical agents.

Although various exemplary embodiments of the invention have been disclosed, it should be apparent to those skilled in the art that various changes and modifications can be made which will achieve some of the advantages of the invention without departing from the true scope of the invention.

What is claimed is:

1. An implantable device comprising:
an implantable cochlear electrode carrier for electrically stimulating cochlear tissue; and
a flexible lubricant coating on the outer surface of at least a portion of the electrode carrier for reducing friction between the electrode carrier and epithelium.

2. An implantable device according to claim 1, wherein the coating is based on a polymer containing a wetting agent for maintaining flexibility of the electrode carrier.

3. An implantable device according to claim 2, wherein the polymer includes at least one of hyaluronic acid, hydroxyethyl cellulose (HEC), carboxymethyl cellulose (CMC), hydroxyethyl starch, polyethylene glycol (PEG) and carbomer.

4. An implantable device according to claim 2, wherein the wetting agent includes at least one of glycerol and propylene glycol.

5. An implantable device according to claim 1, wherein the coating is biodegradable.

6. An implantable device according to claim 1, wherein the portion of the electrode carrier is the apical end.

7. An implantable device according to claim 1, wherein the portion of the electrode carrier includes the entire insertable portion of the electrode carrier.

8. An implantable device according to claim 1, wherein thickness of the coating varies along the length of the electrode carrier.

9. An implantable device according to claim 1, wherein the coating includes a therapeutically effective amount of a pharmaceutical agent for surrounding tissue of the inner ear

10. An implantable device according to claim 9, wherein concentration of the pharmaceutical agent varies along the length of the electrode carrier.

11. An implantable device according to claim 9, wherein the pharmaceutical agent includes at least one of an immunosuppressive component and an anti-proliferative component.

12. An implantable device according to claim 9, wherein the coating includes a first region having a first pharmaceutical agent and a second region having a second pharmaceutical agent.

13. A lubricant coating for an implantable medical device, the coating comprising:
a flexible lubricant component adapted for coating the outer surface of at least a portion of a cochlear implant electrode carrier for reducing friction between the electrode carrier and epithelium.

14. A lubricant coating according to claim 13, wherein the flexible lubricant component is based on a polymer containing a wetting agent for maintaining flexibility of the electrode carrier.

15. A lubricant coating according to claim 14, wherein the polymer includes at least one of hyaluronic acid, hydroxyethyl cellulose (HEC), carboxymethyl cellulose (CMC), hydroxyethyl starch, and carbomer.

16. A lubricant coating according to claim 14, wherein the wetting agent includes at least one of glycerol and propylene glycol.

17. A lubricant coating according to claim 13, wherein the coating is biodegradable.

18. A lubricant coating according to claim 13, further comprising:
a therapeutic component including therapeutically effective amount of a pharmaceutical agent for surrounding tissue of the electrode carrier when implanted.

19. A lubricant coating according to claim 18, wherein the pharmaceutical agent includes at least one of an immunosuppressive component and an anti-proliferative component.

20. A lubricant coating according to claim 18, wherein the coating includes a first therapeutic component having a first pharmaceutical agent and a second therapeutic component having a second pharmaceutical agent.
